SEQUENCE LISTING (7 sequences)

SEQ ID NO: 1 - protein, 101 aa, Medicago truncatula
MASKQQNRQELEEKAKQGETVVPGGTGGKSLEAQEHLAEGRSKGGQTRKEQLGTEGYQEMGRKGGLSTMEKSGGERAEEEGIDIDESKFKTGGGGGRSQNK

SEQ ID NO: 2 - protein, 14 aa, Medicago truncatula
  VARIANT (2)..(2): H or R
GXSXGGXTRXXQXG

SEQ ID NO: 3 - protein, 14 aa, Medicago truncatula
  VARIANT (1)..(1): D or E
XQXXXXGYXXMGXX

SEQ ID NO: 4 - protein, 14 aa, Medicago truncatula
GRSKGGQTRKEQLG

SEQ ID NO: 5 - protein, 14 aa, Medicago truncatula
EQLGTEGYQEMGRK

SEQ ID NO: 6 - protein, 14 aa, Medicago truncatula
  VARIANT (2)..(2): H or R
GXSXGXXTRXXQXG

SEQ ID NO: 7 - protein, 14 aa, Medicago truncatula
  VARIANT (1)..(1): D, Q or E
XQXXXXGYXXMXXX

(19) United States
(12) Patent Application Publication
(10) Pub. No.: US 2010/0285508 A1
(43) Pub. Date: Nov. 11, 2010

(54) USE OF THE PROTEIN EM6 AS A PERFORMANCE MARKER FOR GERMINATION OF SEED LOTS AND APPLICATIONS THEREOF

(76) Inventors: Julia Buitink, Ingrandes (FR); Olivier Leprince, Ingrandes (FR)

Correspondence Address: MORGAN LEWIS & BOCKIUS LLP, 1111 PENNSYLVANIA AVENUE NW, WASHINGTON, DC, 20004, US

(21) Appl. No.: 12/669,386

(22) PCT Filed: Jul. 17, 2008
(86) PCT No.: PCT/FR08/01053

§ 371 Date: Mar. 11, 2010

(30) Foreign Application Priority Data
Jul. 17, 2007 (FR) 0705164

(51) Int. Cl. G01N 33/53; C07K 16/16

(52) U.S. Cl. 435/7.92; 530/387.9; 436/501

(57) ABSTRACT

The invention relates to the determination of the level, maintenance and/or performance of germination of a seed lot, by quantification of the protein EM6, belonging to the group of Late Embryogenesis Abundant (LEA) proteins. The present invention further relates to antibodies permitting the quantification of the protein EM6.

The present invention relates to the use of the EM6 protein as a marker for the germination performance of seed lots.

One of the challenges for the seed industry is to obtain a seed lot which has a high germination vigor, i.e. a good ability to germinate rapidly and uniformly, but also a high growth quality of the plants which are derived therefrom, irrespective of the environmental and sowing conditions.

Several germination steps in nondormant seeds have been identified. The imbibition phase I is characterized by a passive influx of water which occurs rapidly in the dry seed in a state of latent life. Next, a phase II in which the rate of imbibition is reduced is followed by a final phase in which, once again, the seed absorbs water. It is over the course of this final step that elongation of the radicle takes place, resulting in germination, i.e. protrusion of said radicle out of the teguments of the seed. During these three phases, the metabolism is gradually reactivated. Moreover, over the course of the imbibition phases I and II, the seed remains capable of surviving any drying which brings its water content to values similar to those observed in a non-germinated seed. This property is called desiccation tolerance. During drying, the cellular and metabolic events which result in germination are arrested and the seed returns to a state of latent life. The desiccation tolerance is gradually lost during imbibition, first in the radicle when it has pierced the teguments, and then subsequently in the other parts of the embryo.

In plants, the presence of LEA (Late Embryonic Abundant) proteins is correlated with desiccation tolerance. In particular, these proteins accumulate during maturation of the seed (Cuming, 1999, Kluwer Academic Press, the Netherlands, pp. 753-780) and they are assumed to have a role of protection against the effects of desiccation and other osmotic stresses, both in the vegetative tissues and in the seeds.

They are classified in 5 groups according to their sequence homology. More specifically, a correlation exists between decreased amounts of certain LEAs of groups 1, 2, 3 and 5 and the loss of desiccation tolerance during germination (Boudet et al., 2006, Plant Physiology, 140: 1418-1436).

The EM6 protein belongs to LEA protein group 1. Boudet et al. (Plant Physiology, 2006, 140: 1418-1436) have identified this protein in Medicago truncatula. The polypeptide sequence of this protein is accessible on Genbank under number ABB13462 (Boudet et al., Plant Physiology, 2006, 140: 1418-1436). They have shown that it is particularly expressed in desiccation-tolerant radicles, before emergence thereof. Furthermore, after germination, at the 2.8 mm protrusion stage, the expression of this protein in the radicles that have become sensitive is increased by an osmotic treatment with polyethylene glycol (PEG) which reestablishes the desiccation tolerance.

EM6 homologs have been identified in many other higher plants: by way of nonlimiting examples, mention will in particular be made of Arabidopsis thaliana (Genbank AAD25932, NP181546, Swissprot Q02973, Q07187), Glycine max (Swissprot P93165), Vigna radiata (Genbank AAB07225), Oryza sativa (Swissprot P46520), Triticum aestivum (Swissprot Q9ZR71), Hordeum vulgare (Swissprot P46532), Secale cereale (Swissprot Q9M4P1), Zea mays (Swissprot P46517), Raphanus sativus (Swissprot P11573), Brassica napes (Swissprot O65725), Lycopersicum esculentum (TAIR Gene Index TC178231), Phaseolus aureus (Swissprot Q41685), Vitis vinifera (TrEMBL A5BF16), Daucus carota (Swissprot Q5KTS6), Helianthus annuus (Swissprot P46514).

Many methods for treating seeds (pre-germination, priming), aimed at activating seed germination with a view to improving germination performance before marketing, are well known. The seeds thus treated are more vigorous, germinate and/or shoot more rapidly or more successfully under disadvantageous conditions. It is essentially a question of producing pre-germinated seeds, i.e. seeds in which the radicle has not yet emerged, by means of a controlled treatment of imbibition by hydration, followed by drying. The imbibition can be carried out with an osmoticum such as PEG or salts (KNO₃) or without osmoticum. The seeds are subsequently dried, thereby reducing their water content to values similar to those observed for non-germinated seeds.

Certain pre-germination methods are based on the treatment of seeds at more advanced stages, after radicle emergence (EP 0 202 879). However, at this stage, the seed is much more fragile.

Methods exist which use a hydration step and then the addition of a gel (Agrigel) in water or of an encapsulating polymer (U.S. Pat. No. 4,780,987).

In addition to the pre-germination treatments, post-harvest conditioning methods comprising imbibition steps can be applied to the seeds. These are coating or film-coating methods or methods of treatment with chemical substances. These conditioning methods can also be applied independently of the pre-germination treatment.

The pre-germination treatment should be carried out under conditions which make it possible, firstly, to initiate the germination homogeneously and, secondly, to stop it sufficiently early for it to be possible for the seed to be capable of effectively re-initializing the germination. There is therefore a need to precisely determine the imbibition treatment conditions, in particular the duration of the treatment, in order to meet the seed quality criteria. If it is too short, no beneficial effects on the germination performance are observed. At worst, there is no germination initiation. If the duration of the seed imbibition treatment is too long, this has the effect of reducing the longevity and the germination performance of the dried treated seeds, which makes their storage and the management of the seed stocks more problematic, or even impossible.

Furthermore, if the treatment is carried out poorly and fails (by virtue of the very heterogeneous nature of the seed lots or following a handling error), the lot is lost. It is therefore important to be able to monitor the imbibition treatment in order to be able, if necessary, to adapt it during the process.

The physiological quality of the seed lot can in particular be defined in terms of germination performance and/or of storage longevity and also of degree of heterogeneity of the lot.

The germination performance (or T(50)) of a seed lot is estimated, for example, by the germination rate, expressed in hours, in order to reach 50% germination after sowing under precise conditions defined for each species or variety according to ISTA (International Seed Testing Association) standards. Other methods exist, published in the literature, such as measurements of germination capacity under hydric stresses (Bettey and Finch-Savage, 1996, Seed Science Research, 6: 165-173).

The storage longevity (or P50) is defined by the aging time for which 50% germination is obtained for a seed lot at a precise and controlled relative humidity and temperature, for example 75% relative humidity and 35° C. This storage longevity test is known by seed producers to be an indicator of germination performance (Ibrahim et al., 1993, Journal of seed technology, 17: 29-37 and Ellis and Roberts, 1981, Seed Science and Technology, 9: 373-409).

However, the time required to carry out the tests indicated above is several days to several weeks and is often longer than the pre-germination treatment in itself, which is from 3 to 7 days.

In order to bypass this problem, molecular markers which make it possible to monitor the pre-germination have been characterized. However, either the method for using the marker remains laborious to carry out (by measurements of enzymatic activity or of soluble-sugar content (Bettey and Finch-Savage, 1996, Seed Science Research, 6: 165-173; EP 0 202 879)), or the marker in itself is not specific for the seed.

The inventors have noted, during the imbibition of Medicago truncatula seeds, a significant linear correlation between, on the one hand, the EM6 content, measured using an antibody directed against said EM6 protein, in an extract of soluble proteins of an unemerged radicle and, on the other hand, the storage longevity of the seeds concerned. They also observed that the linearity coefficient between the amount of EM6 and the storage longevity remains stable even when various imbibition conditions are applied to the seeds.

The inventors have, in addition, noted that the antibody directed against Medicago truncatula EM6 recognizes the homologous proteins in the seeds or embryos of other species of dicotyledons and of monocotylendons, and that the linear correlation between the amount of protein recognized by this antibody in the unemerged radicle and the storage longevity is also observed in these other species.

It therefore appears that the EM6 protein is a robust marker which is perfectly correlated with the storage longevity and which therefore makes it possible to monitor the change in the latter during imbibition treatments on a seed lot.

The inventors have also identified, on the basis of the EM6 protein of Medicago truncatula, two peptides which make it possible to obtain antibodies capable of recognizing EM6 homologs in other plant species, and which can be used like the antibody directed against the whole EM6 protein, for quantifying this protein, and monitoring the change therein during an imbibition treatment.

The subject of the present invention is a method for determining the storage longevity of a seed lot undergoing an imbibition treatment, characterized in that it comprises the quantification, in a sample of seeds taken from said lot, of the proteins recognized by an antibody chosen from:

a) an antibody which recognizes a protein, hereinafter called EM6 protein, exhibiting at least 70%, preferably at least 75% and entirely preferably at least 80% identity, or at least 80%, preferably at least 85% and entirely preferably at least 90% similarity with the Medicago truncatula EM6 protein identified by GenBank accession number ABB13462 (and also represented in the appended sequence listing under number SEQ ID NO: 1), and containing a region defined by the general sequence (I) GX₁SX₂GGX₃TRX₂X₄QX₅G (SEQ ID NO: 2) where X₁ represents H or R, X₂ represents K or R, X₃ represents Q or N, X₄ represents E or D, X₅ represents L or M, or by the general sequence (Ia) GX₁SX₂GX₃X₄TRX₂X₅QX₆G (SEQ ID NO: 6) where X₁ represents H or R, X₂ represents H, K or R, X₃ represents G or A, X₄ represents Q, H, E or N, X₅ represents E, Q or D, X₆ represents L, I or M, and a region defined by the general sequence (II) X₁QX₂X₃X₄X₅GYX₆X₅MGX₇X₈ (SEQ ID NO: 3) where X₁ represents D or E, X₂ represents L or M, X₃ represents G or S, X₄ represents T, E or Q, X₅ represents E or Q, X₆ represents K, H, Q, S or R, X₇ represents R or K and X₈ represents K or Q, or by the general sequence (IIa) X₁QX₂X₃X₄X₅GYX₆X₅MX₇X₈X₉ (SEQ ID NO: 7) where X₁ represents D, Q or E, X₂ represents L, I or M, X₃ represents G or A, X₄ represents S, T, G, E, H, K or Q, X₅ represents E or Q, X₆ represents K, H, Q, S or R, X₇ represents G or A, X₈ represents R, K, P or H and X₉ represents K or Q;

b) an antibody directed against a portion of said EM6 protein comprising at least one of the sequences (I), (Ia), (II) or (IIa).

According to one preferred embodiment of the method of the present invention, an antibody directed against a portion of said EM6 protein comprising at least one of the sequences GRSKGGQTRKEQLG (SEQ ID NO: 4) or EQLGTEGYQEMGRK (SEQ ID NO: 5) is used.

Unless otherwise specified, the percentage identities and percentage similarities to which reference is made herein are calculated using the BLAST software (Altschul et al., 1997, Nucleic Acids Res., 25: 3389-402), with the default parameters.

The method in accordance with the invention can be implemented over the course of an imbibition step which can take place during a post-harvest conditioning and/or a pre-germination treatment or any other treatment resulting in a modification of the water content of the seed or of a part of the seed, either over time, or in terms of its distribution within the tissues of the seed.

The term “imbibition treatment” is intended to mean any treatment containing at least one seed hydration step, which can be carried out either in the presence of an inert osmoticum such as PEG or a salt (for example, KNO₃) or else without osmoticum (for example, hydro-conditioning, soaking). Among the imbibition treatments, mention may in particular be made of pre-germination treatment, and various methods of post-harvest conditioning of the seed, for instance coating or film-coating. The hydration methods may be varied and comprise, for example, the use of steam, bringing the seeds into contact with a liquid film or soaking the seeds.

The graphic representation of the correlation between the amount of EM6 detected by the method in accordance with the invention and the storage longevity and/or the germination performance can be obtained on the basis of at least 3 points, each corresponding to a seed lot having undergone imbibition treatments of different durations. The linearity coefficient may be directly deduced therefrom, which subsequently makes it possible to quantitatively determine the storage longevity, whatever the imbibition treatment subsequently applied.

Those skilled in the art may, by quantifying the EM6 protein content of the sample, for example compare the effects of various operating conditions of a given treatment on the storage longevity or the germination performance. They may also compare the effects of an imbibition treatment over the course of time.

The method in accordance with the invention may advantageously be implemented during the pre-germination treatment in order to determine the duration of the treatment for obtaining a seed lot with a desired storage longevity. Since the amounts of EM6 protein change in a linear manner with the storage longevity, the variations in amounts of EM6 protein observed are equal to the variations in storage longevity.

Throughout the treatment, seed samples are taken in order to determine the EM6 content. The treatment is stopped when an EM6 content corresponding to the desired storage longevity value is observed.

For example, for old seed lots, a short imbibition results in an increase in the EM6 content.

If those skilled in the art do not know the value of the linearity coefficient of the seed lot or variety or species, they may, prior to the treatment, themselves determine, on a sample of seeds to be treated, the value of the linearity coefficient as defined above.

The method in accordance with the invention may be implemented on whole seeds, or on embryos or unemerged radicles isolated beforehand from the seeds.

Advantageously, the method according to the invention may be implemented separately on individual seeds taken from the test lot; this makes it possible to determine the degree of heterogeneity of a seed lot for the storage longevity.

The subject of the present invention is also any anti-EM6 antibody directed against a region of said EM6 protein chosen from the region defined by the sequences (I) or (Ia) and the region defined by the sequences (II) or (IIa) above.

An antibody directed against a region of an EM6 protein is defined herein as any antibody capable of detectably binding with the region concerned, on the whole EM6 protein or on a fragment thereof, but not detectably binding with another region of the same protein. In the case of monoclonal antibodies, this includes any antibody which recognizes an epitope located in said region; in the case of polyclonal antibodies, this includes any preparation of antibodies not exhibiting significant cross reactions with other regions of the EM6 protein.

On the other hand, this antibody will advantageously exhibit cross reactions with the homologous region of EM6 proteins of other plant species. In particular, as indicated above, antibodies directed against the region defined by the sequence (I) or by the sequence (Ia) generally have a very broad specificity and can recognize a large variety of EM6 proteins in monocotyledons or dicotyledons, whereas the antibodies directed against the region defined by the sequence (II) or by the sequence (IIa) generally have a more restricted specificity.

Antibodies in accordance with the invention can be obtained by methods well known per se. Conventionally, a fragment of EM6 protein (in the form of a natural, recombinant or synthetic peptide) comprising the region against which it is desired to direct the antibody, or at least one fragment of this region of sufficient size to constitute a B epitope (generally at least 5 to 7 amino acids) is used as immunogen. If necessary, said peptide is mixed with an adjuvant, or coupled to a carrier protein, in order to increase its immunogenicity. The antibodies obtained can then be purified, in a manner also known per se. Generally, this purification comprises at least one affinity chromatography step on a column to which the peptide against which the antibody should be directed is grafted. It is also possible to use the whole EM6 protein as immunogen and to carry out an affinity chromatography on a column to which the peptide against which the antibody should be directed is grafted.

By way of nonlimiting examples of anti-EM6 antibodies directed against a region defined by the general sequence (I), mention may be made of antibodies directed against the peptide defined by the sequence GRSKGGQTRKEQLG (SEQ ID NO: 4).

By way of nonlimiting examples of anti-EM6 antibodies directed against a region defined by the general sequence (II), mention may be made of antibodies directed against the peptide defined by the sequence EQLGTEGYQEMGRK (SEQ ID NO: 5).

The subject of the present invention is also a method for quantifying EM6 in plant material, characterized in that it comprises bringing said plant material into contact with an anti-EM6 antibody in accordance with the invention, as defined above.

The subject of the present invention is also the use of an anti-EM6 antibody in accordance with the invention for determining the storage longevity of a seed lot.

The present invention will be understood more clearly from the further description which follows, which refers to examples illustrating the demonstration of the linear correlation between the amount of EM6 and the storage longevity.

### EXAMPLE I

### Correlation Between the Storage Longevity and the Amount of EM6 Determined by ELISA in Medicago Truncatula

Imbibition Treatment and Priming

A lot of Medicago truncatula (var Parragio) seeds is divided into units of 100 seeds which are imbibed in a 9 cm Petri dish either on a Whatman filter paper No. 1 imbibed with 4 ml of distilled water or on two Whatman filter papers No. 1 imbibed with 7 ml of a solution of polyethylene glycol (Sigma, Mw 6000-8000) equivalent to hydric potentials calculated from the formula of Michel and Kaufmann, 1973 (Plant Physiology 51: 914-916). The Petri dishes are subsequently placed in the dark at 20° C. for various times. Next, the seeds are optionally rinsed rapidly with running water in order to remove the PEG. They are rapidly dried in a ventilated chamber at 43% relative humidity at 20° C. in the dark for 3 days.

Aging Treatment

The untreated seeds or the seeds hydrated beforehand and then dried are deposited on a grid in a hermetically sealed dish containing a relative humidity of 75%. Said grid is obtained by depositing a saturated solution of NaCl at the bottom of the dish. The seeds are thus incubated at 35° C. in the dark for increasing times. Over time, 100 seeds are removed from the dish and germinated in distilled water as described above. After 7 days, the number of germinated seeds is counted. A seed is considered to have germinated when the radicle has emerged out of the teguments of the seed.

The storage longevity is expressed in days required to obtain 50% germination for each batch of aged seeds P(50).

Protein Extraction and ELISA

The protocol for preparing the protein extracts from the Medicago truncatula radicles and that for the ELISA assays are described in Job et al., Seed Science Research, 1997: 225-243. The proteins present in the soluble extracts are then assayed according to the Bradford method (by means of the Biorad Protein Assay kit, Biorad). The following biological samples were prepared according to this method:
radicles isolated from whole seeds imbibed without osmoticum respectively for 3, 7, 11 and 15 hours at 20° C. in the dark;
radicles isolated from whole seeds imbibed for 1 day and 3 days in an aqueous solution of PEG at a hydric potential of −1 MPa and at −1.5 MPa at 20° C. in the dark.

The samples can be prepared in a standard 96-well ELISA microtitration plate (Greiner Bio-One ref 655161). The soluble protein extracts obtained by means of the method described above are diluted in PBS buffer (150 mM NaCl, 10 mM Na₂HPO₄) to 100 ng/μl. 2 μl of these dilutions are added to 248 μl of PBS buffer. Next, successive two-fold dilutions are prepared from the most concentrated extract, taking care to thoroughly homogenize the mixtures.

A volume of 100 μl of each concentration for each sample is transferred into an ELISA microtitration plate. The plate with the samples is incubated for 30 minutes at 35° C., and then washed 5 times with 125 μl of (PBS, 0.05% Tween-20).

The rabbit anti-EM6 primary antibodies directed against the whole recombinant EM6 protein of Medicago truncatula, or anti-EM6-peptide II directed against the sequence EQLGTEGYQEMGRK (SEQ ID NO: 5) of the EM6 protein of Medicago truncatula are diluted to 1/1000 in PBS buffer. 100 μl of antibody solution are deposited per well. The plate is incubated for 30 minutes at 35° C., and then washed 5 times with 125 μl of buffer (PBS+0.05% Tween-20). The peroxidase-coupled secondary antibodies (Anti-Rabbit IgG Alkaline Phosphatase, Sigma) are diluted to 1/10 000 in PBS alone. 100 μl of antibody solution are deposited per well. The plate is then incubated for 30 minutes at 35° C., and then washed 5 times with 125 μl of (PBS+0.05% Tween-20).

The microplate is developed by adding a solution containing the substrates for the abovementioned peroxidase (ABTS, Sigma). 100 μl of ABTS developer are deposited per well. The reaction is monitored on a spectrophotometer at 405 nm in a microplate reader, the intensity of the coloration being proportional to the amount of antigen in the extract of soluble proteins. The absorbance is measured every 5 to 10 minutes, for 1 h. To determine the value of the relative EM6 content, the slope of the regression line of the absorbance values as a function of increasing concentrations of protein extracts is calculated.

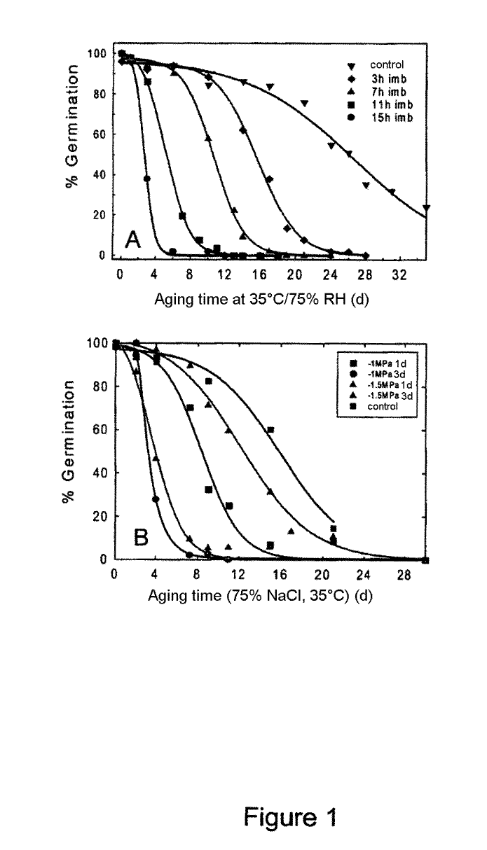

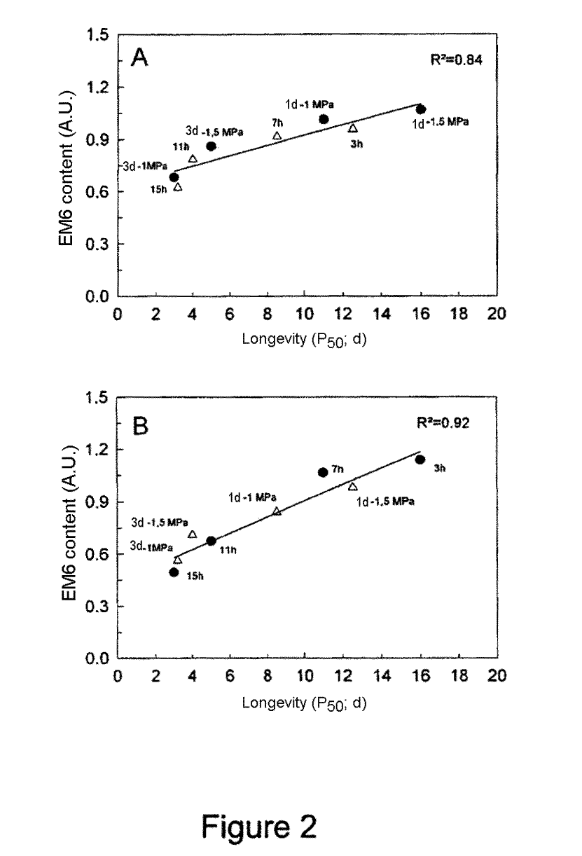

The results are illustrated by FIGS. 1 and 2.

Legend of FIG. 1:

A: % germination at 20° C. as a function of aging time during storage at 35° C., 75% RH, subsequent to an imbibition treatment in distilled water of increasing duration. The control corresponds to nonimbibed seeds.

B: % germination at 20° C. as a function of aging time during storage at 35° C., 75% RH, subsequent to an imbibition treatment in the presence of an osmoticum (polyethylene glycol) corresponding to two hydric potentials (−1 MPa and −1.5 MPa) for 1 or 3 days. The control corresponds to nonimbibed seeds.

Legend of FIG. 2:

Correlation between the amount of EM6 and the longevity capacity P(50) on lots of Medicago truncatula seeds imbibed in distilled water in the presence of osmoticum (PEG) (circles) and without osmoticum (triangles). The imbibition treatment modes are 3, 7, 11 and 15 hours of imbibition in water; 1 or 3 days in a solution of PEG corresponding to a hydric potential of −1.5 or −1.0 MPa. The amount of EM6 is determined in extracts of radicle soluble proteins by ELISA with, on the one hand, a rabbit anti-EM6 antibody directed against the whole recombinant Medicago truncatula EM6 protein (A) and, on the other hand, the anti-EM6-peptide II antibody directed against the sequence EQLGTEGYQEMGRK (SEQ ID NO: 5) of the Medicago truncatula EM6 protein (B).

The control represents non-germinated seeds. FIG. 1 shows that, independently of the imbibition time, the percentage germination measured after 7 days of imbibition decreases as a function of the aging time. FIG. 1 makes it possible to determine the storage longevity (P50) for each treatment. It is noted that the more the imbibition treatment time increases, the more the value of P(50) decreases, whatever the type of imbibition treatment applied (in distilled water (A) or in an aqueous solution of PEG (B)).

FIG. 2 shows that a significant linear relationship exists between the EM6 content, determined by means of an ELISA assay with two different antibodies, and P(50), obtained as a function of the imbibition treatment modes. Furthermore, the correlation coefficient is conserved whatever the type of imbibition treatment used (with or without osmoticum). The ELISA assay set up therefore makes it possible to distinguish Medicago truncatula seeds that have been pretreated according to their storage longevity.

### EXAMPLE II

### Correlation Between the Germination Performance and the Amount of EM6 in Meidcago Truncatula

The protein samples extracted from radicles isolated from treated seeds are prepared according to the protocol described in example 1. In order to determine the imbibition rates (T50), the treated seeds are dried as described in example 1. Next, 100 seeds are imbibed at 20° C. as described in example 1. The seeds having germinated are counted every 2 hours. The germination percentages are transferred onto a graph as a function of imbibition time in order to determine the time necessary to obtain 50% germination for each seed lot (T20).

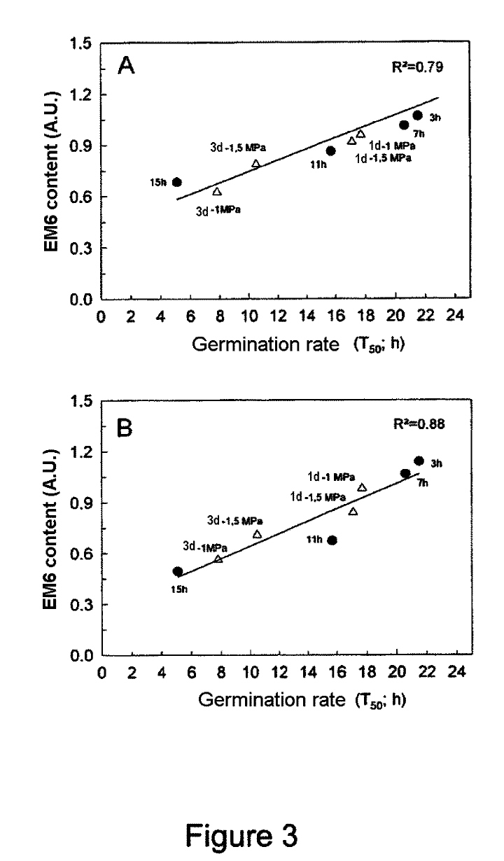

The results are summarized in FIG. 3.

Legend of FIG. 3:

Correlation between the amount of EM6 and the germination performance, measured by the germination rate T(50) on lots of Medicago truncatula seeds imbibed in distilled water in the presence of osmoticum (PEG) (triangles) and without osmoticum (circles). The imbibition treatment modes are: 3, 7, 11 and 15 h of imbibition in water; 1 or 3 days in a solution of PEG corresponding to a hydric potential of −1.5 or −1.0 MPa. The amount of EM6 is determined in extracts of radicle soluble proteins by means of an ELISA assay with, on the one hand, a rabbit anti-EM6 antibody directed against the whole recombinant Medicago truncatula EM6 protein (A) and, on the other hand, the anti-EM6-peptide II antibody directed against the sequence EQLGTEGYQEMGRK (SEQ ID NO: 5) of the Medicago truncatula EM6 protein (B).

Beforehand, it was possible to verify that, the more the imbibition treatment time increases, the more the T(50) decreases. The results of FIGS. 3A and B show that there is indeed a significant linear correlation between the T50 and the EM6 content determined by means of an ELISA assay in the presence of two different antibodies. Thus, the EM6 content is closely correlated with the germination vigor of the lots.

### EXAMPLE III

### Reactivity of the Anti-EM6 Antibodies on Protein Extracts of Dicotyledonous and Monocotyledonous Seeds

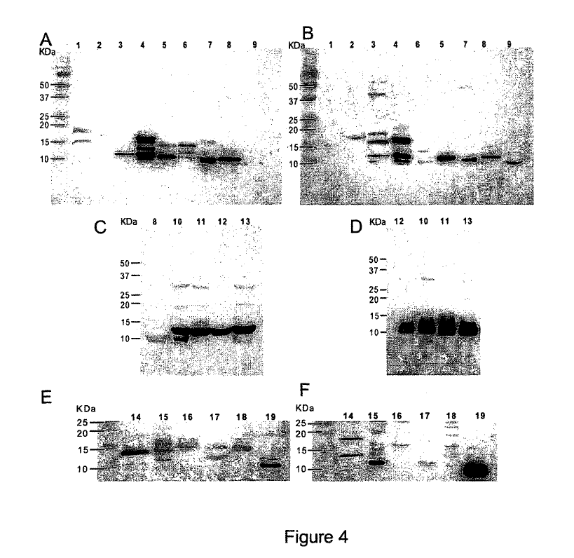

Other experiments were carried out on seed lots from other species in order to determine whether the anti-EM6-peptide I and anti-EM6-peptide II antibodies recognized EM6 homologs in these other species. The results are summarized in FIG. 4.

Legend of FIG. 4:

A+C: Specific antigen-antibody recognition by the Western blotting technique using soluble protein extracts of seeds of 13 species (lane 1 to 13) by means of the anti-EM6-peptide II antibody directed against the sequence EQLGTEGYQEMGRK (SEQ ID NO: 5) of the Medicago truncatula EM6 protein. Beforehand, 2.5-30 μg of proteins, according to the species, were separated by gel electrophoresis according to their molecular weight (molecular marker indicated on the left lane) and transferred onto a nitrocellulose membrane. The assigning of the lanes to each species and the corresponding protein concentrations are the following: 1, celery (25 μg of protein); 2, carrot (25 μg); 3, cucumber (30 μg); 4, leek (15 μg); 5, chive (15 μg); 6, onion (15 μg); 7, busy lizzie (15 μg); 8, petunia (15 μg); 9, pansy (15 μg); 10, radish (2.5 μg); 11, broccoli (2.5 μg); 12, rapeseed (2.5 μg); 13, cabbage (2.5 μg).

B+D: Specific antigen-antibody recognition by the Western blotting technique using soluble protein extracts of seeds of 13 species (lane 1 to 13) by means of the anti-EM6-peptide I antibody directed against the sequence GRSKGGQTRKEQLG (SEQ ID NO: 4) of the Medicago truncatula EM6 protein. The assigning of the lanes and the protein concentrations for each species are identical to those mentioned above.

E+F: Specific antigen-antibody recognition by the Western blotting technique using soluble protein extracts of radicles isolated from 7 species, by means of the anti-EM6-peptide II antibody (E) and the anti-EM6-peptide I antibody (F). The assigning of the lanes to each species and the corresponding protein concentrations are the following: 14, Medicago truncatula (30 μg); 15, black-eyed pea (15 μg); 16, soybean (15 μg); 17, pea (15 μg); 18, bean (15 μg); 19, corn (15 μg).

The protein samples extracted from dry whole seeds or from radicles isolated from dry seeds are prepared according to the protocol described in example 1. After electrophoresis on a 12% (w/v) polyacrylamide gel, the gels are equilibrated for twice 10 min in a transfer buffer (8 mM Tris HCl [pH 6.8], 61 mM glycine, 20% (v/v) methanol). The polyvinylidine difluoride (PVDF) or Immobilon membranes (Millipore) are incubated for 10 seconds in pure methanol and then equilibrated in the transfer buffer. The semi-dry transfer (Biorad) is carried out under a current of 15V for 15 minutes. The detection is carried out according to Boudet et al., 2006, Plant Physiology, 140: 1418-1436. The primary anti-EM6-peptide I and peptide II antibody is diluted to 1/10 000. The developing is carried out by chemiluminescence using the Immun-Star Western C kit (Biorad), under the conditions recommended by the manufacturer. The image is taken with the Chemidoc imager (Biorad) after optimization of the exposure of the signal according to the conditions recommended by the manufacturer.

These results show that there is a cross-reaction between the antibodies directed against the peptide sequences of the Medicago truncatula EM6 protein and the EM6 homologs of dicotyledons or of monocotyledons. For most of the species, one or two major bands are detected by the two antibodies. These results show the specificity of the antibody and the existence of several common epitopes in a significant amount.

### EXAMPLE IV

### Correlation Between the Storage Longevity of Sunflower Seeds and the Amount of EM6 Determined by ELISA

Experiments were carried out in order to validate, on sunflower, the correlation between the abundance of EM6 and the germination performance after imbibition treatments with or without osmoticum (PEG).

Imbibition Treatment and Priming

A lot of sunflower seeds is divided into units of 80 seeds which are imbibed in the presence of distilled water or an aqueous solution of PEG as described in example I. The seeds are then placed in the dark at 20° C. for increasing times (0 h, 6 h and 15 h in water and 48 h in the PEG solution at −1.5 MPa). Next, they are optionally rapidly rinsed with running water in order to remove the PEG and then rapidly dried in a ventilated chamber at 43% relative humidity at 20° C. in the dark for 3 days.

Aging Treatment

The untreated seeds or the seeds hydrated beforehand and then dried are aged according to the protocol described in example I. During storage, 80 seeds are removed successively and germinated under conditions described in example I. After 7 days, the number of germinated seeds is counted. The storage longevity is measured by the P(75), defined by the aging time necessary to obtain 75% germination for the seed lot.

Protein Extraction and ELISA Assay

The protocol for preparing the protein extracts from isolated radicles and the quantification of the proteins are described in example I. The preparation of the samples for the ELISA assay is described in example I, apart from the fact that the successive two-fold dilutions are carried out starting from a protein concentration of 100 ng/μl. The procedure described in example I was monitored using the anti-EM6-peptide II antibody directed against the sequence EQLGTEGYQEMGRK (SEQ ID NO: 5) of the Medicago truncatula EM6 protein.

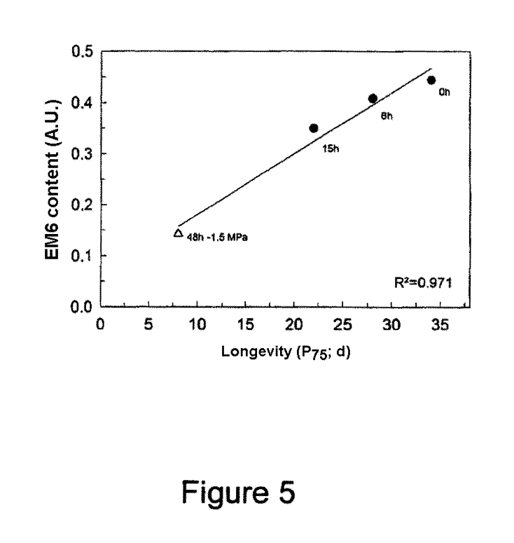

The results are illustrated by FIG. 5.

Legend of FIG. 5:

Relationship between the relative amount of EM6 (determined by ELISA assay using soluble protein extracts of isolated radicles by means of the anti-EM6-peptide II antibody) and the longevity capacity of sunflower seeds having undergone various imbibition treatments. The seeds were imbibed for increasing times (indicated on the figure) in distilled water (circles) or 48 h in the presence of an aqueous solution of PEG corresponding to a hydric potential of −1.5 MPa (circles). The longevity capacity corresponds here to the time necessary to obtain 75% germination after aging after storage at 35° C., 75% RH.

Used in ELISA assays as described for Medicago truncatula, the anti-EM6-peptide II antibody gives, in sunflower, the same result as in the examples described above. In particular, FIG. 5 shows that a linear correlation (correlation coefficient=0.971) exists between the relative amount of EM6 and the longevity capacity P(75) obtained for the various imbibition treatments. The more the longevity decreases, the more the relative EM6 content decreases proportionally, whatever the type of imbibition treatment used. Moreover, these results also indicate that it is not imperative to carry out an aging experiment that is sufficiently long to attain the value of P50.

### EXAMPLE V

### Correlation Between the Amount of EM6 Determined by ELISA and the Improvement in Storage Longevity of an Aged Onion Seed Lot by Soaking in Distilled Water

One of the objectives of a pre-germination imbibition is to eliminate the effects of aging undergone by a lot of seeds that are too aged or stored under poor conditions. Experiments were carried out to show that EM6 is correlated with the longevity capacity when the latter changes as a function of the soaking time in aged onion seeds.

Imbibition and Aging Treatment

A naturally aged onion seed lot is divided up into units of 80 seeds and imbibed according to the protocol described in example I. Next, the seeds are rapidly dried in a ventilated chamber at 43% relative humidity at 20° C. in the dark for 3 days. The procedure described in example I is followed for the seed aging treatment.

Protein Extraction and ELISA Assay

The protocol for preparing the protein extracts and the quantification of the proteins are described in example I. The preparation of the samples and the ELISA assay are described in example I, apart from the fact that the successive two-fold dilutions are carried out starting from a protein concentration of 1 μg/μl. In this experiment, the anti-EM6-peptide II antibody directed against the EQLGTEGYQEMGRK sequence (SEQ ID NO: 5) of the Medicago truncatula EM6 protein was used.

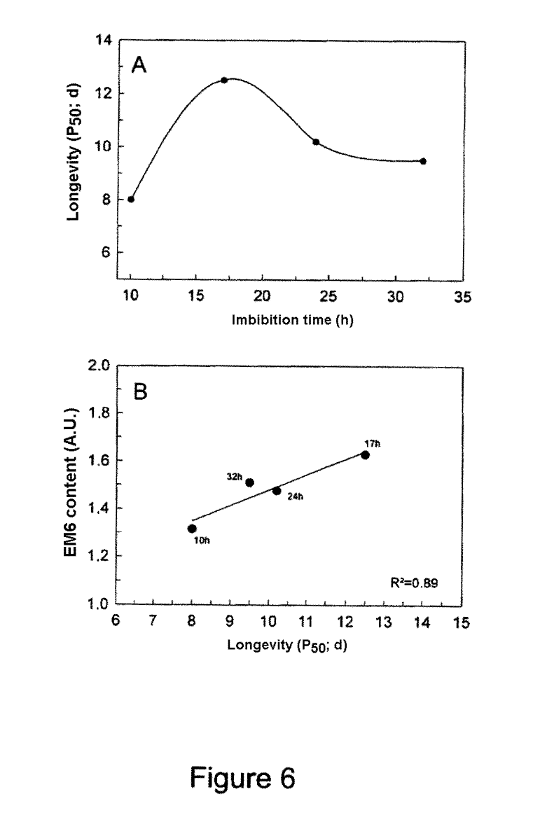

The results are illustrated by FIG. 6.

Legend of FIG. 6:

A. Effect of the duration of imbibition in distilled water, at 20° C. in the dark, of naturally aged onion seeds on the longevity capacity (P50).

B. Relationship between the relative amount of EM6, determined by ELISA assay by means of the anti-EM6-peptide II antibody directed against the sequence EQLGTEGYQEMGRK (SEQ ID NO: 5) of the Medicago truncatula EM6 protein and the longevity capacity P(50) according to increasing imbibition times (indicated on the figure).

FIG. 6A shows that, between 10 and 17 hours of imbibition, the value of P50 increases from 8 to 12.5 days. Next, it decreases after 24 h of imbibition and stabilizes around a P50 value equivalent to 9-10 days. In FIG. 6B, the graph shows the EM6 content as a function of all the P50 values obtained after increasing imbibition times. It is clearly observed that a linear relationship exists between the relative amount of EM6 in the radicles and the longevity capacity.

### EXAMPLE VI

### Correlation Between the Amount of EM6 Determined by Western Blotting and Image Analysis and the Improvement in the Storage Longevity of a Lot of Broccoli Seeds

It is not always possible (because of a lack of time or an insufficient amount of seeds) to carry out an exhaustive study of the loss of germination capacity during aging, as described in example 1. The experiment below shows that the EM6 content predicts the longevity capacity of broccoli seeds when a single aging point is performed. It also shows that the determination of EM6 can be carried out by Western blotting combined with signal quantification by image analysis.

Imbibition Treatment and Aging

A lot of broccoli seeds is divided up into units of 100 seeds and imbibed according to the protocol described in example I. The Petri dishes are then placed in the dark at 20° C. for increasing periods (6 h and 15 h in water and 24 and 48 h in the solution of PEG corresponding to a hydric potential of −1.5 MPa). Next, the seeds are optionally rapidly rinsed with running water in order to remove the PEG. They are rapidly dried in a ventilated chamber at 43% relative humidity at 20° C. in the dark for 3 days. The untreated seeds or the seeds hydrated beforehand and then dried are then stored in a relative humidity at 35° C. according to the protocol described in example I. After 19 and 25 days of aging, 100 seeds are germinated under conditions described in example I. After 7 days, the number of germinated seeds is counted.

Protein Extraction, Western Blotting and Signal Quantification

The protocol for preparing the protein extracts from isolated radicles and the quantification of the proteins are described in example I. One μg of protein extracts is separated by electrophoresis on a 12% (w/v) polyacrylamide gel as described in example II and transferred onto an immobilon polyvinylidine difluoride membrane (Millipore). The semi-dry transfer (Biorad) is carried out under a current of 15V for 15 minutes. The EM6 is detected according to Boudet et al., 2006, Plant Physiology, 140: 1418-1436. The anti-EM6-peptide I primary antibody directed against the sequence GRSKGGQTRKEQLG (SEQ ID NO: 4) of the Medicago truncatula EM6 protein is diluted to 1/10 000. The developing is carried out by chemiluminescence using the Immun-Star Western C kit (Biorad) under the conditions recommended by the manufacturer. The image is taken and the signal is quantified by means of an imager (Chemidoc, Biorad) after optimization of the exposure according to the conditions recommended by the manufacturer.

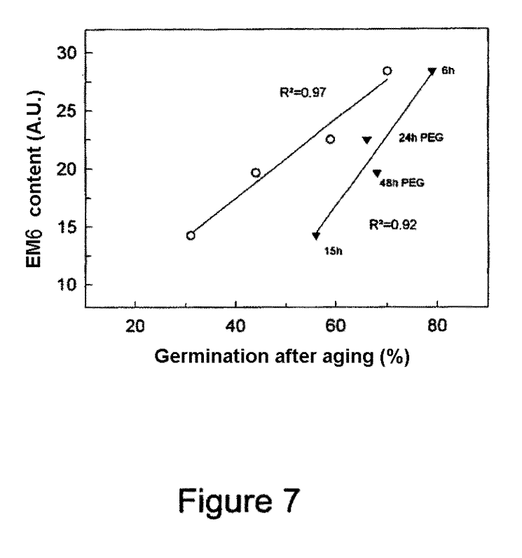

The results are illustrated by FIG. 7.

Legend of FIG. 7:

Relationship between the relative amount of EM6 in radicles isolated from broccoli seeds having undergone various imbibition treatments and the germination percentages obtained after 19 (triangle) and 25 (circle) days of aging at 35° C., 75% RH. Each symbol represents various imbibition modes which are indicated on the figure. EM6 was quantified by image analysis after Western blotting by means of the anti-EM6-peptide I antibody.

The longevity capacity of seeds having undergone various imbibition treatments was measured after 19 (triangles) and 25 (circles) days of aging at 75% RH and 35° C. FIG. 7 shows that a linear relationship (correlation coefficient=0.92 and 0.97) exists between the longevity capacity and the relative amount of EM6 in the broccoli radicles: the more the germination performance measured by means of a single aging point decreases, the more the relative EM6 content decreases proportionally, whatever the type of imbibition treatment used. The results also show that the technique of Western blotting and image analysis can advantageously substitute for the ELISA assay.

1. A method for determining the storage longevity of a seed lot undergoing an imbibition treatment, characterized in that it comprises the quantification, in a seed sample taken from said lot, of the proteins recognized by an antibody chosen from:
a) an antibody which recognizes a protein, hereinafter called EM6 protein, exhibiting at least 70%, preferably at least 75% and entirely preferably at least 80% identity, or at least 80%, preferably at least 85% and entirely preferably at least 90% similarity with the Medicago truncatula EM6 protein GenBank ABB13462 (SEQ ID NO: 1), and containing a region defined by the general sequence (I) GX₁SX₂GGX₃TRX₂X₄QX₅G (SEQ ID NO: 2) where X₁ represents H or R, X₂ represents K or R, X₃ represents Q or N, X₄ represents E or D, X₅ represents L or M, or by the general sequence (Ia) GX₁SX₂GX₃X₄TRX₂X₅QX₆G (SEQ ID NO: 6) where X₁ represents H or R, X₂ represents H, K or R, X₃ represents G or A, X₄ represents Q, H, E or N, X₅ represents E, Q or D, X₆ represents L, I or M, and a region defined by the general sequence (II) X₁QX₂X₃X₄X₅GYX₆X₅MGX₇X₈ (SEQ ID NO: 3) where X₁ represents D or E, X₂ represents L or M, X₃ represents G or S, X₄ represents T, E or Q, X₅ represents E or Q, X₆ represents K, H, Q, S or R, X₇ represents R or K and X₈ represents K or Q, or by the general sequence (IIa) X₁QX₂X₃X₄X₅GYX₆X₅MX₇X₈X₉ (SEQ ID NO: 7) where X₁ represents D, Q or E, X₂ represents L, I or M, X₃ represents G or A, X₄ represents S, T, G, E, H, K or Q, X₅ represents E or Q, X₆ represents K, H, Q, S or R, X₇ represents G or A, X₈ represents R, K, P or H and X₉ represents K or Q; or
b) an antibody directed against a portion of said EM6 protein comprising at least one of the sequences (I), (Ia), (II) or (IIa).

2. The method as claimed in claim 1, characterized in that use is made of an anti-EM6 antibody chosen from:
an antibody directed against a peptide of sequence GRSKGGQTRKEQLG (SEQ ID NO: 4);
an antibody directed against a peptide of sequence EQLGTEGYQEMGRK (SEQ ID NO: 5).

3. An anti-EM6 antibody chosen from:
an antibody directed against a peptide defined by the general sequence SEQ ID NO: 2;
an antibody directed against a peptide defined by the general sequence SEQ ID NO: 6;
an antibody directed against a peptide defined by the general sequence SEQ ID NO: 3;
an antibody directed against a peptide defined by the general sequence SEQ ID NO: 7.

4. The anti-EM6 antibody as claimed in claim 3, chosen from:
an antibody directed against the peptide of sequence SEQ ID NO: 4;
an antibody directed against the peptide of general sequence SEQ ID NO: 5.

5. A method for quantifying EM6 in plant material, characterized in that it comprises bringing said plant material into contact with an anti-EM6 antibody as claimed in claim 3.

6. The use of an anti-EM6 antibody as claimed in claim 3, for evaluating the storage longevity of a seed lot
